(19) United States
(12) Patent Application Publication
(10) Pub. No.: US 2020/0222174 A1
(43) Pub. Date: Jul. 16, 2020

(54) CLOSED FUNNEL FOR THE DELIVERY OF A PROSTHETIC IMPLANT

(71) Applicant: Proximate Concepts LLC, Fort Lee, NJ (US)

(72) Inventor: Paul Rosenberg, Saddle River, NJ (US)

(21) Appl. No.: 16/246,983

(22) Filed: Jan. 14, 2019

(51) Int. Cl. A61F 2/12; A61B 34/10; A61F 2/00

(52) U.S. Cl. CPC A61F 2/12; A61B 34/10; A61B 17/3468; A61L 2430/04; A61F 2/0059

(57) ABSTRACT

A surgical device for assisting in the placement of a prosthetic implant within a surgical pocket of a patient. The surgical device according to one embodiment comprises one or more sheets of polymer shaped in the form of a conical frustum in which a proximal end of the frustum is sealed and a distal end of the frustum forms a tip, the interior cavity of the frustum sized to hold a prosthetic implant. A lumen extends into the interior of the frustum to allow the injection of fluid into the interior of the conical frustum and thereby activate any surface coatings applied thereto.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### FIELD OF THE INVENTION

The inventions described herein generally relate to devices to assist in the placement of prosthetic implants. Embodiments of the invention further relate to devices that hold a prosthetic implant within a structure that is (or is generally) in the shape of a closed conical frustum holding a prosthetic implant and with a lumen extending into the interior of the frustum to allow the injection of a fluid thereinto. Cutting an opening the distal end of the conical frustum provides an egress path for the prosthetic implant such that the application of pressure laterally along the length of the frustum ejects the prosthetic implant through the opening and into a surgical pocket that a surgeon opens in the body of a patient. Movement of the prosthetic implant out of the frustum and across one or more surface active coatings causes interaction between the prosthetic implant and one or more surface active coatings, thereby proving the benefits that the one or more surface active coatings offer.

### BACKGROUND OF THE INVENTION

Prosthetic implants, in particular breast implants, have a substantial history dating back to the first recorded implant surgery done in 1895 at the University of Heidelberg, Germany. Instead of being for cosmetic purposes, the physician (Dr. Vincent Czerny) performed the procedure by taking fat from the hip of a female for implantation into her breast, which needed reconstruction after the removal of an adenoma. Accordingly, the purpose of this first “implant” was to reconstruct a breast after devastating surgery, rather than to achieve uniform and/or aesthetic enlargement.

The first real foreign substance injected into body to achieve enlargement is perhaps the injection of paraffin, which comes in a plurality of textures and is primarily composed of petroleum jelly. Dr. Robert Gesurny, an Austrian surgeon who used the material on the testicle of a soldier in an attempt to achieve a more desirable size, first discovered the use of paraffin for boosting the size of bodily objects. Inspired by the results, Dr. Gesurny went on to use the material for breast enlargement injections, universally resulting is severely compromised outcomes.

Modern silicon gel implants, which are well known to those of ordinary skill in the art today, have a long history that dates back over the past seventy years. In fact, some evidence supports Japanese prostitutes after World War II injecting their breasts with various substances, including liquid silicon. The serious worry with silicon in liquid form was that it broke apart, forming granulomas that could then migrate basically anywhere in the body. Liquid silicone is still used, but very rarely, in microscopic amounts, and only using medical-grade silicone that is completely sterile; it remains highly controversial and is associated with serious complications. Breast implants as presently know in the art—silicon as a cohesive mass inside of a polymer “bag” structure—had their debut in the 1960s, developed by Doctor Thomas Cronin and his colleague Frank Gerow.

Although removed from the market for a number of years due to a variety of concerns and complications, silicon was allowed back into breast implants in 2006, albeit in a new form. The FDA, after years of study and experiments, finally allowed silicone gel-filled implants onto the U.S. market, which are the sole breast implant option outside of saline-filled prostheses. Today's silicone gel is designed to feel a bit like human fat: very thick, very sticky, and classified as “semi-solid” or a “cohesive gel”. Such technology actually represents the fifth generation of silicon implant: the first one developed by Cronin and Gerow, with various innovations, including more secure coatings, thicker gels and more natural shapes.

When any type of implant is inserted into a surgical pocket that a surgeon opens in the body of a patient, the body reacts by forming a protective lining around the prosthetic. This biological structure is referred to as the “capsule”, “tissue capsule” or “scar capsule”, though it should be noted that the structure is not exactly the same as scar tissue. Although, a capsule formation is normal and happens regardless of whether the implant is smooth or textured, silicone or saline-filled, some theorize that problematic capsule growth is potentially attributed to manipulation of tissue during implant placement. In addition, trauma to tissue along or adjacent to the surgical pocket may suffer from the direct formation of scar tissue due to potentially over aggressive manipulation of tissue at the entry site for the implant along the edge of the surgical pocket. Moreover, recent studies have called into question the role of bioactive substances, collectively referred to as “bioburden”, as a potential root cause of capsular contracture and which may include colonization and subclinical infection by bacterial or other bioactive organisms. Such diverse bioactive organisms are readily found on the skin surface and/or within the breast gland and ductal tissue desquamated cells, all of which may be drawn into the breast implant cavity by manipulation and insertion of prostheses.

Accordingly, what is needed in the art are new devices that allow for minimal contact with a prosthetic implant prior to placement of a prosthetic implant while avoiding the unnecessary manipulation of tissue adjacent to or inside a surgical pocket, which serves to minimize the development of scar tissue and the formation of any significant capsule around the prosthetic implant. What is further needed in the art are such devices with surface active coatings that minimize complications that develop during, as well as subsequent to, placement of a prosthetic implant, e.g., infection, capsular contracture, etc.

### SUMMARY OF THE INVENTION

Embodiments of the invention are directed towards a surgical device for assisting in the placement of a prosthetic implant within a surgical pocket of a patient. According to one embodiment, the surgical device comprises one or more sheets of polymer shaped in the form of a conical frustum. The frustum is sealed with an implant disposed therein and a lumen extending into the interior of the frustum. Such an arrangement provides for a sealed or closed conical frustum that contains the prosthetic implant and having a lumen through which a surgeon can inject fluid thereinto to allow for the activation of any coatings applied to the interior surface of the conical frustum. Additional embodiments comprise replacing the lumen with a conduit containing an injection port that may be embedded within the exterior wall of the conical frustum, or a tube containing a remote injection port that interfaces with the exterior wall of the conical frustum.

Excision of the distal tip of the frustum provides an egress path allowing the surgeon to place the prosthetic implant into a surgical pocket that he or she opens in the patient. The configuration of the surgical device of the present invention allows for the device to accept and place of a variety of prosthetic implant types including, but not limited to, a breast implant, a pectoral implant, a calf implant and a gluteal implant. In a given instance, the interior cavity of the frustum is sufficiently sized to hold the specific type of prosthetic implant that is to be placed within the surgical pocket of the patient.

In addition to the use of a lumen extending into the interior cavity of the conical frustum, other embodiments contemplate the use of a fluid pocket, which may be integrated into the frustum itself or formed contiguously therewith. In accordance with one or more embodiments, a volume at the tip of the frustum located at the far distal end of the frustum is sealed to create a pocket. The seal is a frangible seal (or a seal capable of programmable failure based on the application of a predetermined range of pressure) and the pocket filled with a fluid, for example, sterile saline, such that the application of pressure to the pocket causes the frangible seal to fail and the fluid contents of the pocket enter the main body of the frustum isolated by the frangible seal. As is explained in greater detail herein, entry of fluid into the body of the frustum, either from the fluid pocket or injection via a lumen, serves to activate one or more coatings applied to an interior surface of the conical frustum.

Continuing with the foregoing, a lubricious coating is applied to the interior cavity of the frustum to assist in transferring the prosthetic implant from the interior of the frustum to the surgical pocket of the patient, thereby allowing the prosthetic implant to easily exit the frustum. While certain embodiments utilize a water activated lubricious coating on the interior of the device, the lubricious coating is more generally any hydrophilic gel that decreases the coefficient of friction of the inside surface of the frustum.

According to one embodiment, the frustum is constructed from a polymer selected from the set of polymers consisting of medical grade vinyl, medical grade PVC, medical grade nylon, polyethylene, polypropylene or other synthetic polymer materials from which the conical frustum may be fashioned. When utilizing medical grade vinyl in the construction of the frustum, various embodiments utilize medical grade vinyl that is between 0.01 inches and 0.005 inches thick.

In addition to the lubricious coating, one or more surface active coatings may be applied to the interior cavity of the frustum. Movement of the prosthetic implant across the one or more surface active coatings as it exits the frustum causes interaction between the prosthetic implant and the one or more surface active coatings. Such interaction allows the surface active coatings to provide one or more benefits that such coatings offer, such as exposure of the implant to the antimicrobial effects of a given surface active coating.

The outside of the device may comprise a number of different markings or indicia that aid the surgeon in properly placing the prosthetic implant within the surgical pocket. For example, the device may comprise a marker, such as a circular marker, which allows the surgeon to achieve proper alignment of the prosthetic implant during placement into the surgical pocket. Application of the marker may be in accordance with any number of processes that do not affect the integrity of the material forming the device, such as a silkscreen process. The device may also have a plurality of size guide lines set at varying distances from the opening in at the distal end of the device. The surgeon can use the plurality of size guide lines to properly align a cutting device to excise excess material from the distal opening of the device as well as to remove any structures provided to assist in the irrigation of the interior of the conical frustum, e.g., lumen and port, frangible or burstable saline-filled pouch, injection port, etc.

The lubricious coating on the inside surface of the device is activated by exposure to water, thereby resulting in a decrease in the coefficient of friction of the inside surface of the device. Activation of the lubricious coating may be through injection of water into the interior of the device through the use of a syringe, or aqueous submersion of the device. Alternatively, the surgeon or technician exposes a sheet of absorbable material placed within the device to water, thereby allowing water to wick through the absorbable material and activate a lubricious coating on an inside surface of the device. According to one embodiment, the sheet of absorbable material changes color when exposed to water. In an alternative embodiment, the material comprising the body of the conical frustum maybe porous or microperforated along a portion or the entirely of the exterior surface, thereby allowing for activation of any coatings on the interior surface of the conical frustum by simply submerging the frustum in a bath of aqueous solution

### BRIEF DESCRIPTION OF THE DRAWINGS

The invention is illustrated in the figures of the accompanying drawings which are meant to be exemplary and not limiting, in which like references are intended to refer to like or corresponding parts, and in which:

FIG. 1 illustrates a top perspective view of a surgical device for assisting in the placement of a prosthetic implant within a surgical pocket of a patient according to one embodiment of the present invention;

FIG. 2 illustrates a top perspective view of a surgical device for assisting in the placement of a prosthetic implant within a surgical pocket of a patient according to one embodiment of the present invention;

FIG. 3 illustrates a magnified top perspective view of a surgical device for assisting in the placement of a prosthetic implant within a surgical pocket according to one embodiment of the present invention; and

FIG. 4 illustrates a top perspective view of a surgical device for assisting in the placement of a prosthetic implant within a surgical pocket of a patient according to an alternative embodiment of the present invention.

### DETAILED DESCRIPTION OF THE INVENTION

Subject matter will now be described more fully hereinafter with reference to the accompanying drawings, which form a part hereof, and which show, by way of illustration, exemplary embodiments in which the invention may be practiced. Subject matter may, however, be embodied in a variety of different forms and, therefore, covered or claimed subject matter is intended to be construed as not being limited to any example embodiments set forth herein; example embodiments are provided merely to be illustrative. Those of skill in the art understand that other embodiments may be utilized, and structural changes may be made without departing from the scope of the present invention. Likewise, a reasonably broad scope for claimed or covered subject matter is intended. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to one and only one embodiment. Similarly, the same embodiment and the phrase “in another embodiment” as used herein does not necessarily refer to a different embodiment. It is intended, for example, that claimed subject matter include combinations of example embodiments in whole or in part.

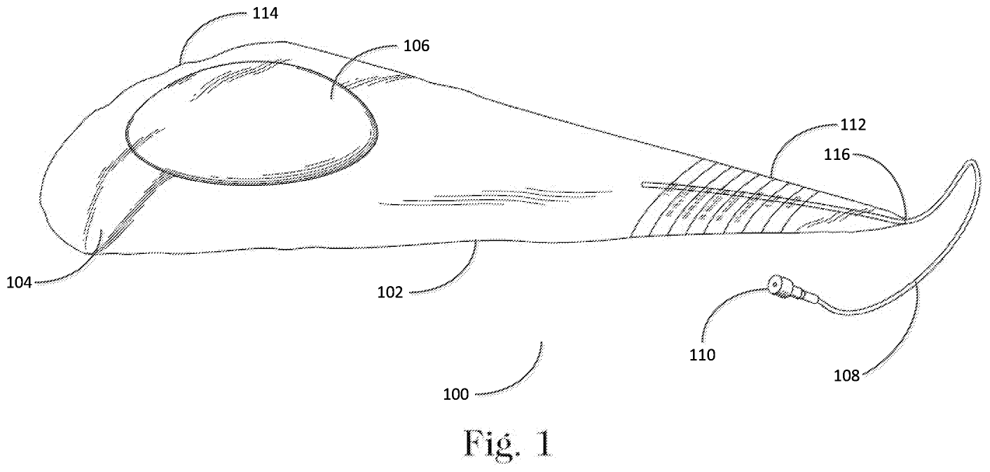

FIG. 1 illustrates a top perspective view of a surgical device 100 for assisting in the placement of a prosthetic implant within a surgical pocket of a patient according to one embodiment of the present invention. The surgical device 100 comprises a bag shaped as a conical frustum in which a proximal end 114 of the conical frustum is sealed and a distal end of the conical frustum forms a tip 116. According to one embodiment, the proximal end 114 of the device 100 is sealed with an RF weld or similar technique including, but not limited to, the application of ultrasonic energy, heat, pressure or the like. Similarly, a seam (not pictured) can be formed by shaping one or more sheets of polymer into the form of a conical frustum and sealed with an RF or other suitable weld. The device 100 may be formed from a single sheet of material that is stamped or cut using a die or other suitable technique and folded around itself, or may be formed by multiple panels, e.g., front and back panels, that are joined together.

The device 100 is accordingly shaped in the form of a sealed conical frustum, which may have a prosthetic 106 implanted disposed in the interior 104 thereof. Extending from the exterior of the device 102 and terminating within the interior cavity 104 of the conical frustum is a lumen 108. The lumen 108 provides an entry point to the closed frustum 100 allowing a surgeon or surgical technician to introduce fluid and thereby irrigate an interior surface 104 of the device 100.

The end of the lumen that terminates external to the conical frustum 100 may be fitted with or have a port 110 affixed thereto. As such, the surgeon or surgical technician may interface a fluid filled syringe (not pictured) with the port 110 to introduce fluid to the interior surface 104 of the conical frustum 100 and thereby irrigate the interior surface 104 of the conical frustum. In the present embodiment, the lumen 108 enters the conical frustum at the distal tip 116 of the conical frustum 100, which may be sealed around the lumen 108. Alternative embodiments contemplate alternative interface points between the lumen 108 and the exterior surface 102 of the conical frustum 100. For example, as an alternative to the lumen 108 entering the interior 104 of the conical frustum from the tip 116, the lumen may interface the conical frustum 100 from any point located on the exterior surface 102 of the conical frustum 100.

The inside surface of the surgical device 100, which is a bag in the form of a conical frustum, is treated with a lubricious coating. Lubricity is a property that describes how slippery a surface is or, more specifically, the value of its coefficient of friction. Embodiments of the present invention benefit from treatment to ensure a slippery surface to reduce the insertion force that the surgeon must apply to propel or eject the prosthetic implant through the distal opening in the device. Benefits include avoiding possible puncture damage to the prosthetic implant and eliminating severe abrasion between the implant surface and the interior surface of the device.

The lubricious coating can be applied to interior surface of the device 100 in a number of ways. One application technique is dip coating, which entails suspending the device from a support, lowering it into a liquid coating solution and then withdrawing. The coating sticks to the surface as the support draws the device up and out of the solution. An alternative is spray coating, which is akin to airbrushing and involves spraying a nebulized mist over the interior surface of the device. Film coating is another type of application process in which long rolls of material that form the device are drawn from reel to reel through a tank of liquid coating. Other processes for the application of the lubricious coating include chemical vapor deposition and silk screening, which are primarily suitable when working with flat surfaces, such as the raw material that forms the device.

Any of the above-described coating techniques involves a drying or curing process to permanently affix the lubricious coating to the interior surface of the device. Two prevalent methods for curing hydrophilic coatings include applying heat and ultraviolet (“UV”) light. When utilizing heat-cured coatings, the device is placed within an oven for a given period of time. Such controlled heating accelerates drying of the coating and any necessary chemical reactions taking place within the coating (or between the coating and the device), such as crosslinking, which allows the coating to adhere to the interior surface of the device, as well as provide durability. For embodiments that utilize lubricious coatings that use UV light for the curing step, the interior surface of the device is exposed to UV light for a given period of time, which has the effect of stimulating any necessary covalent or ionic chemical reactions that the curing process may require. Completion of the curing process affixes the lubricious coating to the interior surface of the device.

The lubricious coating is hydrophilic. Hydrophilic coatings exhibit so-called “water loving” characteristics. From a chemical perspective, these coatings participate in dynamic hydrogen bonding with surrounding water. In some cases, hydrophilic coatings are also ionic, and may further exhibit negative charge, which facilitates additional aqueous interactions. Physically, these chemical interactions with water give rise to hydrogel materials that may exhibit extremely low coefficients of friction. Taken together, these chemical and physical characteristics describe a class of materials that are wettable, lubricious, and suitable for tailored biological interactions.

In addition to the application of a lubricious coating to interior surface of the device 100, a number of disparate surface active coatings may also be applied to the interior surface of the device 100. The use of surface active coatings on the interior surface of the device 100 has advantages over drug eluting covers or coatings that may be applied to the surface of a medical device. One advantage of such surface active coatings over drug eluting coatings is that the surface active coatings do not elute or otherwise release from the interior surface of the device, but rather remain bonded to the interior surface. A further distinction over the use of drug eluting compounds is that the mechanism of action for such surface active coatings is not a specific antibiotic that is targeted towards a specific genus and species of bacteria, but rather a broad based antimicrobial action based on cell membrane rupture of a given virus, bacteria, protozoa, microbe, etc.

According to one embodiment, a given one of the one or more surface active coatings is applied by dipping the device, where the surface active coating is in a liquid phase, in a volume of the given surface active coating. Accordingly, the surface active coating may be simultaneously applied to one or more surfaces of the device. Similarly, where the surface active coating is in liquid form, the surface active coating may be applied to a surface of the device by spraying or airbrushing the surface active coating onto the desired surface. Likewise, vapor depositing and other coating techniques know to those of skill in the art may be used to apply the surface active coating to one or more surfaces of the device. It should also be noted that multiple surface active coatings can be combined in solution for simultaneous application to the desired surfaces.

As with the discussion herein regarding certain lubricious coatings, the surface active coating is cured, if required, which may be by heat, UV light, etc., depending on the curing requirements of the specific surface active coatings. According to one embodiment, components of a first surface active coating are bonded to components of another surface active coating, which is itself then bonded to the surface of the device, both of which may comprise bonding by way of a curing process. Curing allows for bonding of the one or more surface active coatings to bind to the device, which may be ionic or covalent bonds, or combinations thereof, again depending on the specific surface active coatings applied to the surface of the device, as well as any interactions therebetween.

Regardless of the specific type and number of coatings applied to the interior surface 104 of the surgical device 100, such coatings require activation. As described above, fluid is introduced to the interior 104 of the surgical device 100 according to one embodiment by interfacing a fluid source with the lumen, e.g., a fluid filled syringe mating with a port affixed to the distal end of the lumen 108. Pushing the fluid into the interior 104 of the conical frustum 100 causes activation of the coatings affixed thereto.

As indicated, a technician may hydrate the interior of the device 100 to activate any coatings applied thereto by injecting a fluid through the lumen 108 and into the device 100 using a syringe. Alternatively, or in conjunction with the foregoing, the technician may hydrate a polymer insert (not pictured) placed within the interior of the device 100. When hydrating the polymer insert, the insert serves to wick the fluid throughout the interior of the device, thereby activating the lubricious coating. The polymer insert is preferably sized to follow the contours of the interior of the device 100 and may comprise any material that suitably wicks fluid throughout its surface including, but not limited to lightweight stabilizer (60% polyester/40% Rayon), medium weight stabilizer (60% polyester/40% rayon) and medium to heavyweight sew-in interfacing (100% polyester).

The inclusion of a polymer insert inside the conical frustum serves the additional function of isolating the internal surfaces of the frustum when compressed or folded prior to activation of any coatings. This wicking fabric or spacer material may be comprised of paper material or synthetic absorbable fabric and serves to “paint” the internal surface with saline upon removal, thereby evenly distributing the wetting solution upon the interior surface. The insert fabric is removed from the distal tip of the conical frustum following surface coating activation and prior to delivery of the prosthetic implant from the distal tip of the device.

Subsequent to activation of the coatings affixed to the interior surface 104 of the surgical device 100, the surgeon or surgical technician must create an egress path to allow the implant 106 to exit the device 100. One or more guide lines 112 printed on the exterior surface 102 of the device 100 allow the surgeon or surgical technician to create a properly sized opening that allows for egress of the prosthetic implant 106 from the interior cavity of the conical frustum 100. According to one embodiment, a single guide line 112 is printed on the exterior surface 102 of the device 100 that corresponds to the size of the prosthetic implant packaged within the interior cavity of the device. Alternatively, a plurality of guide lines 112 are printed on the exterior surface of the device, a given guide line 112 sized to create an egress aperture corresponding to a respectively sized prosthetic implant.

In an alternative embodiment that does not require physically cutting the distal end along one of the one or more printed guide lines 112, excision of the distal tip may be accomplished according to other embodiments through the inclusion of micro-perforations in the conical frustum, such micro-perforations aligned in any size guide lines printed on the exterior surface of the conical frustum. The use of micro-perforations allows the distal end to be torn and separated along such precut micro-perforations. This allows opening of the device without the use of scissors, which may accidentally cut through any spacing paper or wicking fabric that may be disposed of inside the conical frustum. Once removed, any additional cutting of the distal end of the conical frustum may be achieved using scissors or other cutting instruments to size the distal opening to properly accommodate the implant dimension, texturization and volume.

Excising material from the distal opening of the device allows the technician to increase the aperture diameter of a distal opening that he or she cuts, thereby allowing the device to accommodate various implant sizes without causing damage to the device 100 or prosthetic implant 106. The prosthetic implant exits the device 100 through application of a twisting motion by the surgeon or the sequential application of pressure laterally along the device by the hands of the surgeon. Typically, such twisting motion or sequential application of pressure takes place with the proximate hand of the surgeon applying pressure while the distal hand provides support, although other hand and pressure application configurations fall within the scope of the invention. The application of pressure as such serves to drive the implant out the distal opening formed by the cut and into a surgical pocket that the surgeon has prepared to accept the implant.

As the prosthetic implant 106 moves through the interior of the device towards the egress point at the distal end, the surfaces of the prosthetic implant 106 come into communication with any coatings placed or bonded to the interior surface of the device 100, which then interact with any foreign agents on the exterior surface of the prosthetic implant 106 prior to placement in the surgical pocket. Such communication between the prosthetic implant and the one or more surface active coating allows the coatings to impart one or more benefits that such coatings offer. For example, where the surface active coating has an antimicrobial component, the surface of the prosthetic implant receives the antimicrobial benefits of the surface active coating without any medication or drugs unnecessarily or undesirably eluting into the body of the patient.

The application of such surface active coatings is an advantageous improvement in the functioning of the surgical device in placing a prosthetic implant due to its ability to reduce the occurrence of infection and other complications that arise during and after placement. Embodiments of the invention reduce or eliminate the adhesion of bacteria onto an implantable medical device, such as a prosthetic implant. Adsorption of protein on the outer surface of the implantable device provides a suitable location for bacterial adhesion. Bacterial adhesion on the exterior surface of the prosthetic implant, even in minor amounts, has the possibility of leading to bacterial division, secretion and, ultimately, the development of biofilm across the surface of the prosthetic implant subsequent to its placement inside the patient. Again, the application of surface active coatings to the interior of the device allows the surfaces of the prosthetic implant come into communication with the surface active coatings. This communication allows for the one or more surface active coatings to interact with any foreign agents on the exterior surface of the prosthetic implant prior to placement in the surgical pocket

In an exemplary configuration, the interior surface 104 of the conical frustum 100 has affixed thereto a coating that comprises a non-thrombogenic component and an antimicrobial component. Each of the non-thrombogenic component and antimicrobial component may be bonded to a polymer backbone that is further bonded to the interior surface 104 of the conical frustum. One exemplary surface active coating that can be bonded to the interior surface of the device to provide such non-thrombogenic and antimicrobial effects is the AdvertPlus™ surface active antimicrobial coating available from BioInteractions Ltd.

Beyond the guide lines 112 identified above, different markings on the exterior 102 of the frustum 100 assist in preparing the device for implant delivery and the surgeon in the delivery process. For example, a marker may be printed on the outside 102 of the device 100, which allows the technician to align the prosthetic implant 106 properly in the device 100, such as by aligning the marker with a physical marker on the bottom of the implant. In this manner, the surgeon is made aware of the bottom position of the implant upon delivery and can ensure that the implant is not delivered into the surgical pocket bottom side up. Application of the marker to the device is in accordance with any process that permanently deposits the marker on the outer surface of the device without harming or otherwise causing any degradation to the device itself, e.g., through a silkscreen process, inkjet printing or hand application with suitable pens or markers.

A number of prior art implants are suitable for use with embodiments of the present invention. For example, breast, gluteal and various calf implants, may be set or otherwise placed through use of various embodiments of the device described herein. Those of skill in the art recognize that certain aspects of the frustum geometry might require slight modification from the illustrations presented in conjunction with the embodiments depicted herein. By way of example and not limitation, when placing a gluteal implant, which is typically of a larger volume than a breast implant, the length of the frustum, as well as possibly the angle at which the tip of the frustum is formed, may require commensurate increases.

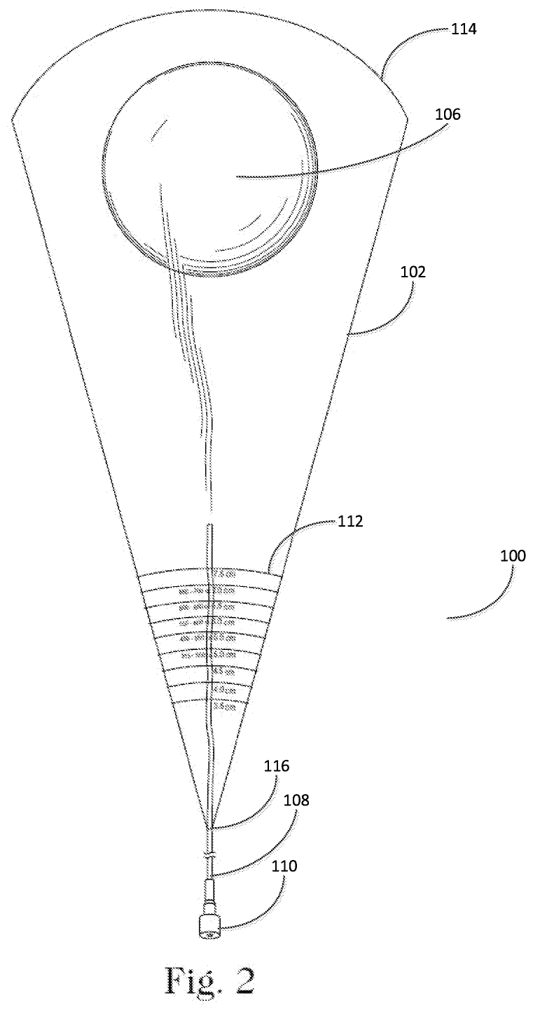

FIG. 2 illustrates a top perspective view of the surgical device of FIG. 1, which is designed for assisting in the placement of a prosthetic implant within a surgical pocket of a patient. As can be seen in the illustration of the surgical device 100 in the view of FIG. 2, the device 100 comprises a bag shaped as a conical frustum in which a proximal end 114 of the conical frustum is sealed and a distal end of the conical frustum forms a tip 116. The device 100 may be formed from a single sheet of material that is stamped or cut using a die or other suitable technique and folded around itself, or may be formed by multiple panels, e.g., front and back panels, that are joined together.

The device 100 is accordingly shaped in the form of a sealed conical frustum, which may have a prosthetic implant 106 disposed in the interior cavity 104 thereof. Extending from the exterior of the device 102 and terminating within the interior cavity 104 of the conical frustum is a lumen 108. The lumen 108 provides an entry point to the device 100 and allows a surgeon or surgical technician to introduce fluid and thereby irrigate an interior surface 104 of the device.

The end of the lumen that terminates external to the device may be fitted with or have a port 110 affixed thereto. As such, the surgeon or surgical technician may interface a fluid filled syringe (not pictured) with the port 110 to introduce fluid to the interior surface 104 of the device and thereby irrigate the interior surface of the conical frustum. In the present embodiment, the lumen 108 enters the conical frustum at the distal tip 116 of the device, which may be sealed around the lumen 108. Alternative embodiments contemplate alternative interface points between the lumen 108 and the exterior surface 102 of the conical frustum 100. For example, as an alternative to the lumen 108 entering the interior of the conical frustum from the tip 116, the lumen may interface the conical frustum 100 from any point located on the exterior surface 102 of the conical frustum 100.

Building on FIGS. 1 and 2, FIG. 3 illustrates a magnified top perspective view of a surgical device for assisting in the placement of a prosthetic implant within a surgical pocket. In the present embodiment, the lumen 108 enters the conical frustum at the distal tip 116 of the conical frustum 100, which may be sealed around the lumen 108. According to various embodiments, the one or more sheets or polymer that form the body of the device 100 are folded into the shape of a conical frustum and sealed around a lumen 108 such that the frustum is sealed closed with the lumen proving an ingress path for the introduction of fluids into the interior of the device. Alternatively, the walls of the conical frustum may be porous or microperforated to permit the introduction of fluids into the interior of the device by submersion of the entire device into a bath of sterile saline, an admixture of sterile saline and antibiotic compounds, or any other desired irrigation fluid.

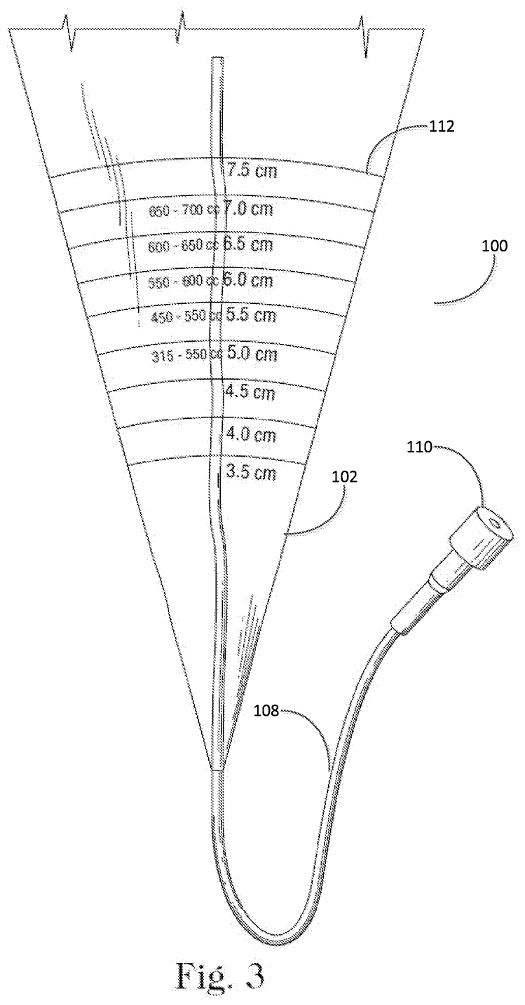

The distal end of lumen 108 according to the embodiment illustrated in FIG. 3 is fitted with or has a port or similar syringe adapter 110 affixed thereto. A surgeon uses a syringe (not pictured) filled with a given volume of fluid and interfaces the syringe with the port 110, for example, a needleless syringe is filled with a volume of sterile saline or, alternatively, a sterile saline and antibiotic admixture. Application of pressure to the plunger of the syringe causes the fluid contained therein to travel the length of the lumen 108, enter the device and coat the interior cavity, thereby activating any coatings on the interior surface. The surgeon or surgical technician excises the distal end of the device to open an egress path allowing the prosthetic implant to exit the device and be deposited within a surgical pocked formed in the body of the patient. It should be noted in this and other embodiments, the length of the lumen disposed within the interior of the device may be sized to not extend past a guideline intended to be cut based on the size of the prosthetic implant disposed of within the device.

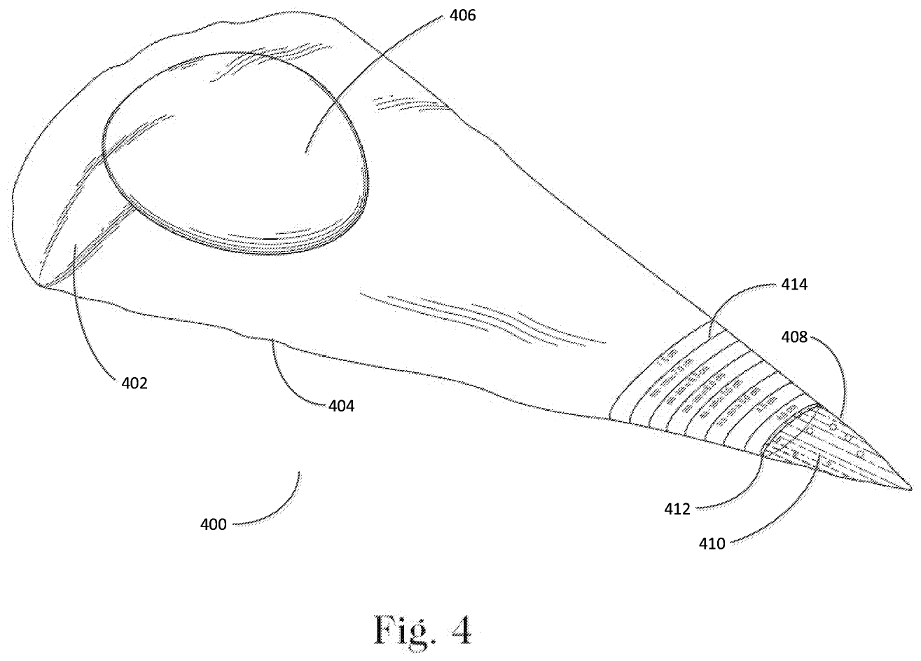

In addition to introducing fluid to the interior cavity of the sealed device using a lumen extending into the interior cavity, other embodiments contemplate the use of a fluid filled pocket, which may be contained within or integrated into the frustum itself. In accordance with one embodiment, FIG. 4 illustrates a surgical device in the form of a conical frustum 400 that comprises a volume 408 at the tip located at the far distal end of the frustum 400 that is sealed 412 to create a pocket. The seal 412 is a frangible seal and the pocket filled with a fluid 410, for example, sterile saline, such that the application of pressure to the volume 408 causes the frangible seal 412 to fail and the fluid contents of the pocket enter the main body 402 of interior cavity the frustum 400 that was previously isolated by the frangible seal 410. As explained above, entry of fluid into the body 402 of the frustum 400, either from the fluid pocket 408 or injection via a lumen, serves to activate one or more coatings applied to an interior surface 404 of the conical frustum.

An egress path for the prosthetic implant must be opened subsequent to breaking the frangible seal 412 and using the fluid 410 contained within the pocket 408 to irrigate the interior surface 402 of the device 400, thereby activating any coatings applied thereto. Accordingly, excision of the distal tip of the frustum, which in various embodiment may be achieved by scissor division or by tearing along a pre-perforated line, provides an egress path allowing the surgeon to place the prosthetic implant into a surgical pocket that he or she opens in the patient. One or more guide lines 414 printed on the exterior surface 404 of the device 400 allow the surgeon or surgical technician to create a properly sized opening that allows for egress of the prosthetic implant 406 from the interior cavity of the conical frustum 400. According to one embodiment, a single guide line 414 is printed on the exterior surface 404 of the device 400 that corresponds to the size of the prosthetic implant packaged within the interior cavity of the device. Alternatively, a plurality of guide lines 414 are printed on the exterior surface 404 of the device 400, a given guide line 414 being sized to create an egress aperture corresponding to a respectively sized prosthetic implant. This allows the technician to increase the aperture diameter of a distal opening that he or she cuts, thereby allowing the device 400 to accommodate various implant sizes without causing damage to the device 400 or prosthetic implant 406.

FIGS. 1 through 4 are conceptual illustrations allowing for an explanation of the present invention. Those of skill in the art should understand that various aspects of the embodiments of the present invention could be implemented using different materials, fasteners and minor design modifications. Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention.

In the present specification, an embodiment showing a singular component should not necessarily be limited to other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

The foregoing description of the specific embodiments will so fully reveal the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present invention have been described above, it should be understood that they have been presented by way of example, and not limitation. It would be apparent to one skilled in the relevant art(s) that various changes in form and detail could be made therein without departing from the spirit and scope of the invention. Thus, the present invention should not be limited by any of the above-described exemplary embodiments, but should be defined only in accordance with the following claims and their equivalents.

What is claimed is:

1. A surgical device for assisting in the placement of a prosthetic implant within a surgical pocket of a patient, the surgical device comprising:
one or more sheets of polymer shaped in the form of a conical frustum in which a proximal end of the frustum is sealed and a distal end of the frustum forms a tip, the interior cavity of the frustum sized to hold a prosthetic implant; and
a lumen extending into the interior of the frustum to allow the injection of fluid into the interior of the conical frustum.

2. The surgical device of claim 1 comprising:
a lubricious coating applied to the interior cavity of the frustum; and
one or more surface active coatings applied to the interior cavity of the frustum, wherein movement of the prosthetic implant out of the frustum and across the one or more surface active coatings causes interaction between the prosthetic implant and the one or more surface active coatings to provide one or more benefits that the one or more surface active coatings offer.

3. The surgical device of claim 2, wherein the lubricious coating is water activated.

4. The surgical device of claim 3, wherein the lubricious coating comprises a hydrophilic gel that decreases a coefficient of friction of the inside surface of the bag.

5. The surgical device of claim 2, wherein a given one of the one or more surface active coatings comprises an antimicrobial coating.

6. The surgical device of claim 2, wherein a given one of the one or more surface active coatings comprises a heparin coating.

7. The surgical device of claim 1, wherein the prosthetic implant is selected from the set of prosthetic implants consisting of a breast implant, a pectoral implant, a calf implant and a gluteal implant.

8. The surgical device of claim 1, wherein the one or more sheets of polymer are between 0.01 inches and 0.005 inches thick.

9. The surgical device of claim 1, wherein the one or more sheets of polymer comprises of one or more sheets of polymer selected from the set of polymers consisting of medical grade vinyl, medical grade PVC, medical grade nylon and polyethylene.

10. The surgical device of claim 1, comprising a marker on the exterior surface of the frustum to properly align the prosthetic implant for placement into the surgical pocket.

11. The surgical device of claim 1, comprising a plurality of size guide lines printed on the one or more sheets of polymer to properly align a cutting device for excision of material from the tip at the distal opening of the frustum and provide an egress path for the prosthetic implant.

12. The surgical device of claim 2 wherein the surface active coating comprises an antimicrobial component.

13. The surgical device of claim 11 wherein the surface active coating comprises a non-thrombogenic component.

14. A surgical device for assisting in the placement of a prosthetic implant within a surgical pocket of a patient, the surgical device comprising:
one or more sheets of polymer shaped in the form of a conical frustum in which a proximal end of the frustum is sealed and a distal end of the frustum forms a tip, the interior cavity of the frustum sized to hold a prosthetic implant;
a frangible seal forming a pocket at the tip of the frustum and isolating the pocket from the remainder of the frustum isolated by the frangible seal; and
a volume of fluid contained within the pocket,
whereby application of pressure to the pocket causes the frangible seal to fail and the fluid contained within the pocket to enter the remainder of the frustum isolated by the frangible seal.
